(19) United States
(12) Patent Application Publication
(10) Pub. No.: US 2013/0233097 A1
(43) Pub. Date: Sep. 12, 2013

(54) Physical and Occupational Therapy Monitoring and Assessment Methods and Apparatus

(71) Applicants: David Alan Hayner, Austin, TX (US); Eric Weidmann, Austin, TX (US)

(72) Inventors: David Alan Hayner, Austin, TX (US); Eric Weidmann, Austin, TX (US)

(21) Appl. No.: 13/787,001

(22) Filed: Mar. 6, 2013

Related U.S. Application Data
(60) Provisional application No. 61/607,655, filed on Mar. 7, 2012.

(51) Int. Cl. A61B 5/11

(52) U.S. Cl. CPC A61B 5/11
USPC 73/865.4

(57) ABSTRACT

Apparatus and methods for the collection, processing, storage, communication and use of data generated by an array of sensors connected to a body for the purposes of monitoring and measuring physical motion. Analysis of the collected data is employed to aid the user in accomplishing more efficient and effective physical exercise or training. Data may also be used by 3^{rd} parties to monitor performance and update training or exercise regimes.

### CROSS-REFERENCE TO RELATED APPLICATIONS

Provisional Utility Patent: Physical and Occupational Therapy Monitoring and Assessment Methods and Apparatus, Provisional application No. 61/607655 filed Mar. 7, 2012

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The present invention is in the technical field addressing applications of sensors. More specifically, this invention discloses the employment of one or more sensors, digital processing systems and storage and communications devices to monitor and assess the physical movements and mechanics of a body performing various training regimes or physical and occupational therapy exercises.

The data collected by a network of sensors can be used to monitor and measure the performance of a body, human or animal, performing various training regimes or the specific exercises required for physical and occupational therapy. The data collected can be processed to quantify the performance of exercises to prescribed regimes. Both the quality and quantity of performance can be measured. Real-time feedback can be provided to the exerciser while they are performing the exercises to aid in recovery or improve training efficiency and effectiveness. Data concerning the exerciser's performance can be collected for review and monitoring. This data can assist both the health or training professional and exerciser in the design and in the execution of specific exercises, regimes, rates and scheduling required to optimize effectiveness. Furthermore, other desirable features and characteristics of the embodiments presented here will become apparent from the subsequent detailed description taken in conjunction with the accompanying drawings and this background.

### SUMMARY OF THE INVENTION

The present invention employs an array of sensors, microprocessors, storage media and communications systems to collect and/or assess data concerning body mechanics of animals performing exercises.

### BRIEF DESCRIPTION OF THE DRAWINGS

Various embodiments will hereinafter be described in conjunction with the following figures, wherein like numerals denote like elements, and

FIG. 1 is a diagram of a network of sensors attached to a human body structure (knee) for the purposes of collecting data regarding the motions of this structure in accordance with one embodiment of the invention;

FIG. 2 is a diagram of a network of sensors, data processing systems, user interface, power supply, storage and communications systems configured in a manner to collect, process, record and communicate data generated from a set of sensors attached to the body in accordance with one embodiment of the invention;

FIG. 3 is a flow diagram of the data collection and analysis processes pertaining to the construction of exercise signatures for the quantification of subsequent exercises in accordance with one embodiment of the invention;

FIG. 4 is a diagram illustrating the structure of a three-dimensional path corresponding to the motion of a specific body structure performing an exercise in accordance with one embodiment of the invention;

FIG. 5 is a flow diagram of the data collection and analysis processes pertaining to the measurement and quantification of exercise motions relative to a previously generated exercise signature in accordance with one embodiment of the invention;

FIG. 6 is a diagram illustrating the structure of a three-dimensional path corresponding to a specific exercise signature and paths representative of a good repetition and flawed repetition of the specific exercise in accordance with one embodiment of the invention;

FIG. 7 is a flow diagram of the data collection process pertaining to the measurement and quantification of exercise motions relative to previously generated exercise signatures and the update of these reference exercise signatures in accordance with one embodiment of the invention;

FIG. 8 illustrates a summary of a basic exercise signature generation scheme and two variations on this method in accordance with one embodiment of this invention;

FIG. 9 illustrates two methods for translating exercise signatures into new exercise signatures in accordance with one embodiment of this invention.

### DETAILED DESCRIPTION OF EMBODIMENTS

The following detailed description is merely exemplary in nature and is not intended to limit the scope or the application and uses of the described embodiments. Furthermore, there is no intention to be bound by any theory presented in the preceding background or the following detailed description.

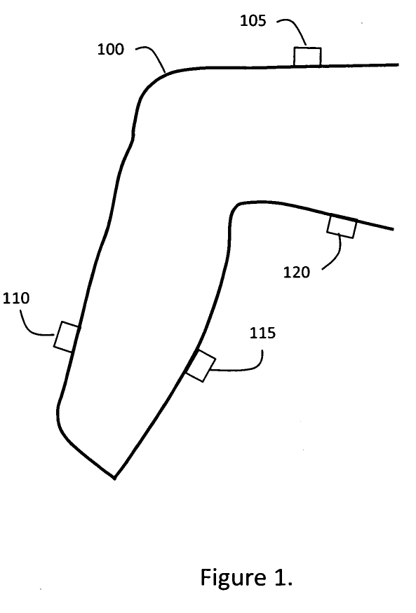

Referring now to the invention, FIG. 1 illustrates multiple sensors, 105, 110, 115 and 120 attached to a human knee 100. These sensors may be attached via adhesive, straps, braces, sleeves or any other method effective for mounting sensor devices on a body in a manner in which they are substantially fixed in position relative to the body. These sensors are arranged in a manner to collect data regarding the motion of the body structure as it moves through specific exercises. This data is used for two purposes. In the first case, this data is used to generate reference exercise signatures describing good and flawed repetitions of the exercise. An external observer, a trainer for instance, can provide the assessments used to quantify a particular repetition of an exercise as good or flawed and/or assign a quality measure. The second use of the data is during user performance of the proscribed exercise to score a specific repetition of the exercise relative to the previously generated exercise signature. This scoring can then be used to measure the quality of each repetition, and count the repetition as successful or not successful in a manner consistent with the assessment the trainer could have provided. In a simple implementation, if a particular repetition of the exercise was sufficiently close to the “good” exercise signature, the system would increment a counter on the user interface or provide other feedback to inform the exerciser that they have successfully completed acceptable repetition of the proscribed exercise. If the particular exercise repetition was not sufficiently close to a “good” exercise signature or too close to a “bad” exercise signature, the system would register this as a flawed repetition and could provide some feedback to the exerciser of this result.

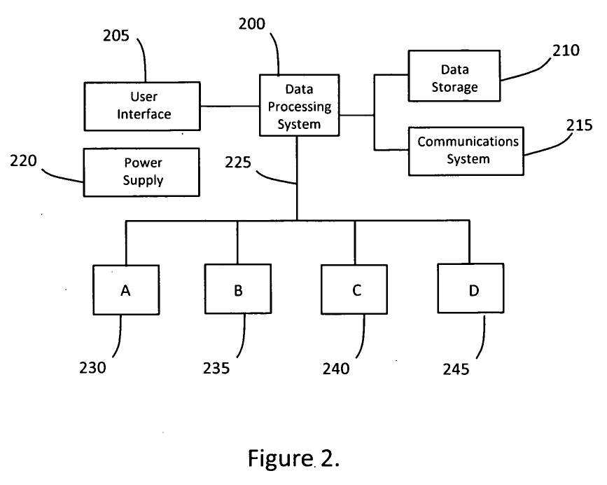

Illustrated in FIG. 2 is a system containing four sensors 230, 235, 240 and 245 connected via some communications bus 225 to data processing system 200. The data processing system 200 is connected to a user interface 205, data storage 210 and communications system 215. A local power supply 220 provides appropriate power for this system. The four sensors 230, 235, 240 and 245 may be physically arranged on a body structure as illustrated in FIG. 1, or in any of a number of alternate physical arrangements or alternate body structures. These sensors measure information regarding the motions of the body structure to which they are attached. This measurement data is collected at some sampling rate by the data processing system 200. In response to software running on the data processing system 200, the sensor data is processed for either the generation of exercise signatures or to measure exercise performance in relation to a specific exercise signature. Results can be communicated to the user and/or trainer via the user interface 205 and/or the communications system 215. Additionally, information regarding the exercise performance can be recorded in the data storage system for later retrieval and analysis.

The sensors 230, 235, 240 and 245 may be any or combination of gyroscopes, linear or angular accelerometers, position encoders, magnetometers, tachometers, strain gauges, pressure sensors, optical or radio frequency measuring systems employed for measuring the movement of the body structure to which these sensors are attached. While this document has referred to four sensors, any number of sensors could be employed in this system without substantially deviating from the methods taught in this patent.

Communications bus 225 and communications system 215 may be any of a number of wireline or wireless systems currently available or may become available in the future. The specifics of these communications devices are substantially independent of the methods taught in this patent.

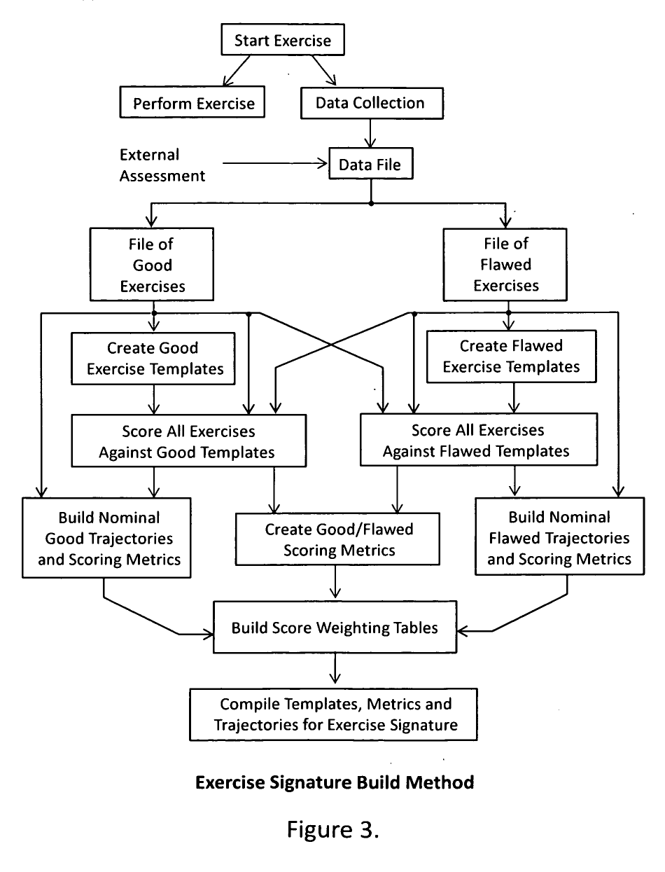

In FIG. 3 is a flow diagram of a training process by which templates for successful and unsuccessful exercises may be generated. The exercise is started and in parallel to the performance of the exercise, data is collected and transferred to the data processing system to create the Data File by the network of sensors as illustrated in FIGS. 1 and 2. An external observer, a trainer for example, monitors this performance of the exercise and via the user interface, 205 in FIG. 2, records a score for each repetition. This is represented by the signal External Assessment in FIG. 3. This assessment may be pass or fail or on some gradated scale. The user repeats this exercise several times with the observer recording an External Assessment as some measure of success or failure for each repetition. This system collects data from some finite number of successful and possibly unsuccessful repetitions together with the assessments or scores. Sensor data plus External Assessment data is combined in Data File in FIG. 3. Data representing these multiple repetitions of a specific exercise, together with the External Assessments are parsed into a File of Good Exercises and a File of Flawed Exercises. In practice, the user interface may also include some means of input allowing the trainer or exerciser to delineate the start and stop of a given repetition of an exercise. This could be a switch, a voice command, an optical queue or some unique motion of the body structure

In the next step, a template generation algorithm is run on each of these data sets to generate one or more reference templates for a good version of the exercise and possibly for flawed, and possibly other grades of exercise quality. In the end, there may be one or more templates representing a good repetition of the exercise or several templates representing various levels of success of the exercise. This process is denoted as Create Good Exercise Templates and Create Flawed Exercise Templates.

The next step is to score all the recorded repetitions of the specific exercise against the set of Good and Flawed templates. These steps are represented by the processes Score All Exercises Against Good Template and Score All Exercises Against Flawed Templates. These scores, together with the a priori knowledge of the quality of each repetition available from the External Assessment data, enables the construction of scoring metrics by which various characteristics of the data are weighted in a process to determine the quality of the repetition. This process is represented by the Create Good/Flawed Scoring Metrics function.

In parallel to the generation of scoring metrics, data in the File of Good Exercises is analyzed to generate a nominal three-dimensional trajectory for various substructures of the body element to which the sensors are attached. For instance, the motion of the lower section of a human leg and the motion of the upper section of a human leg. Together with the nominal trajectory, various statistical metrics of allowable variations to this trajectory are also generated. This process is represented by the functional block Build Nominal Good Trajectories and Scoring Metrics. The process is also performed on the File of Flawed Exercises and a set of corresponding trajectories and statistical metrics are generated for flawed repetitions of the specific exercise. This process is represented by the functional block Build Nominal Flawed Trajectories and Scoring Metrics. In both of these cases, a priori data concerning the quality of each of the repetitions provided by the External Assessment is employed to aid in the generation of the trajectories and statistical metrics.

In the next step, results from the template analysis and trajectory analysis are combined to build a set of weighting tables and decision logic. These tables and logic are designed to appropriately value both trajectory and template data to generate a final score of the set of repetitions matching the original inputs from the External Assessment. This functional step is denoted as Build Score Weighting Tables.

In the final step, templates, trajectories, scoring metrics, tables and logic data are combined into' a file representing a statistical measure of the set of exercise repetitions and means as assessing subsequent repetitions of this specific exercise. This data file is referred to as an Exercise Signature. This final step is represented by the functional block Compile Templates, Metrics and Trajectories for Exercise Signature. This overall process will be referred to as the Exercise Signature Build Method.

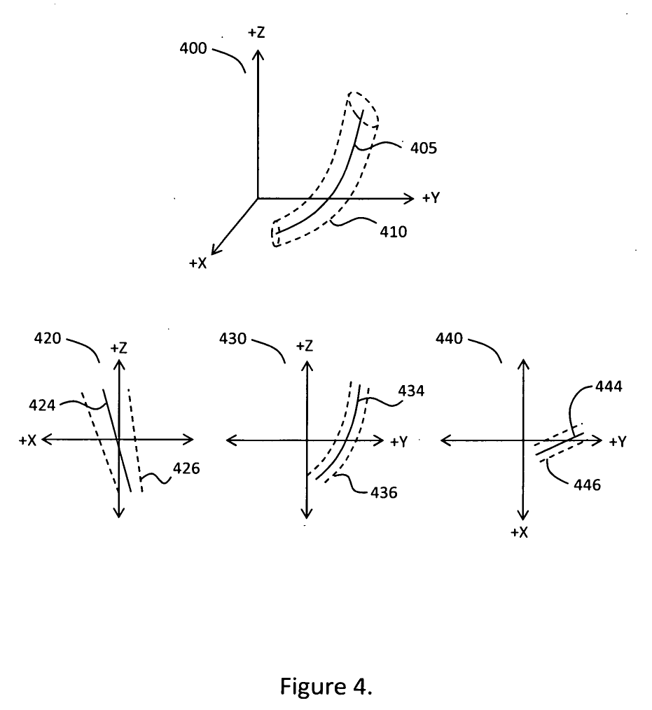

Illustrated in FIG. 4 is set of diagrams illustrating a trajectory in various dimensional views. In 400, a nominal good trajectory is illustrated as the solid line 405. Allowable variations in this trajectory are illustrated with the dashed lines 410 providing an outline of a three-dimensional boundary to this acceptable trajectory. This trajectory may represent, for example, the motion of some specific point on the lower leg of a human during leg extension exercise. Plots 420, 430 and 440 illustrate trajectory 405 and bounds 410 in each of the three planes, XZ, YZ and XY respectively. Trajectory 405 is projected as line 424 in plot 420, as line 434 in plot 430 and line 444 in plot 440. Bounds illustrated as the dashed lines 410 in plot 400 are projected as dashed lines 426 in plot 420, dashed lines 436 in plot 430 and dashed lines 446 in plot 440.

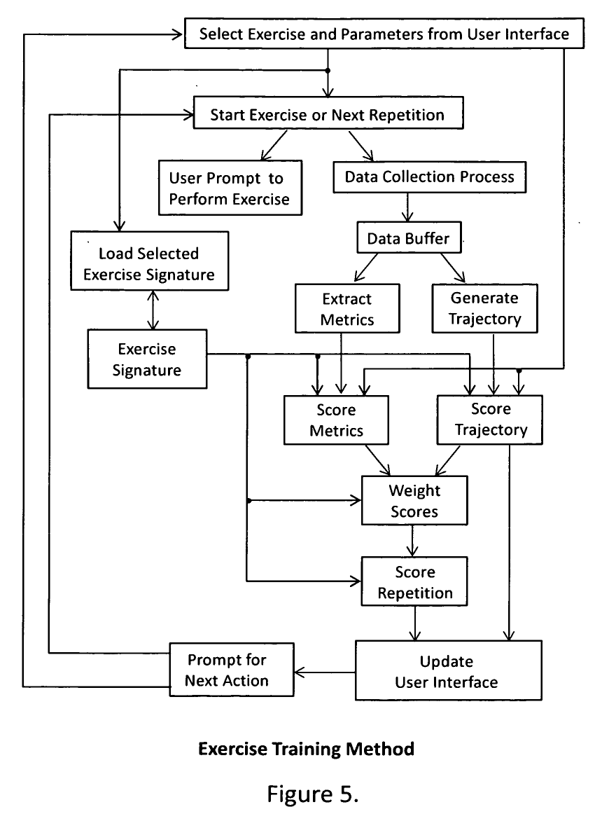

A flow chart representing a process by which exercises are scored in relation to the previously generated Exercise Signature is illustrated in FIG. 5. The user selects a particular exercise via the user interface, 205 in FIG. 2. This is represented as functional block Select Exercise from User Interface in FIG. 5. This action causes the appropriate Exercise Signature to be selected for use. This is indicated by the functional block Load Selected Exercise Signature. The user is prompted to start repetitions of the selected exercise, denoted as User Prompt to Perform Exercise. The system now starts the Data Collection Process, collecting data from the array of sensors. This data is placed in a Data Buffer for use by the Extract Metrics and Generate Trajectory functions. Extracted metrics and the generated trajectory are compared against relevant data sets in the Exercise Signature file in functional blocks Score Metrics and Score Trajectory respectively. These scores are then weighted in the Weight Scores function. This score, together with relevant decision logic criteria from the Exercise Signature file are employed in Score Repetition to determine a final score for this particular repetition. This final score can be as simple as successful or unsuccessful. Alternately, a numeric score can be assigned.

This final score can be communicated via the user interface to the exerciser as indicated in the Update User Interface functional block. This communication may be as simple as a visually indicated count of successful repetitions. Alternately, some combination of an audible, optical, mechanical or electrical feedback concerning success/fail or numeric score of a particular repetition may be implemented. The system may then prompt the user to begin the next repetition, or if a sufficient number of successful repetitions have been completed, or a sufficient total score of repetitions generated, to conclude this session and possibly prompt the user to proceed to the next exercise. This overall process of scoring a repetition of a given exercise to a specific Exercise Signature will be referred to as the Exercise Training Method.

In addition to assigning a score to the repetition, the trajectory of the specific repetition may also be generated and information regarding violations of the bounds on the trajectory can be provided to the user. This feedback may be after the completion of the repetition, or in some cases, during the execution of the repetition to help guide the user in the correct execution of the exercise. This feedback may be audio, graphical, electrical, mechanical or combinations of these methods.

Other capabilities provided in this system are the ability to adjust the scoring tolerances, e.g., allow for more or less variation in a repetition graded as good or flawed. These parameters would be available to the user and/or trainer on the user interface. This is represented in FIG. 5 as the functional block Select Exercise and Parameters from User Interface. Parameters from this selection process influence operations in the Score Metrics and Score Trajectory functions as illustrated in FIG. 5. These parameter selections may well impact other functional blocks as well depending upon specific implementations of this system.

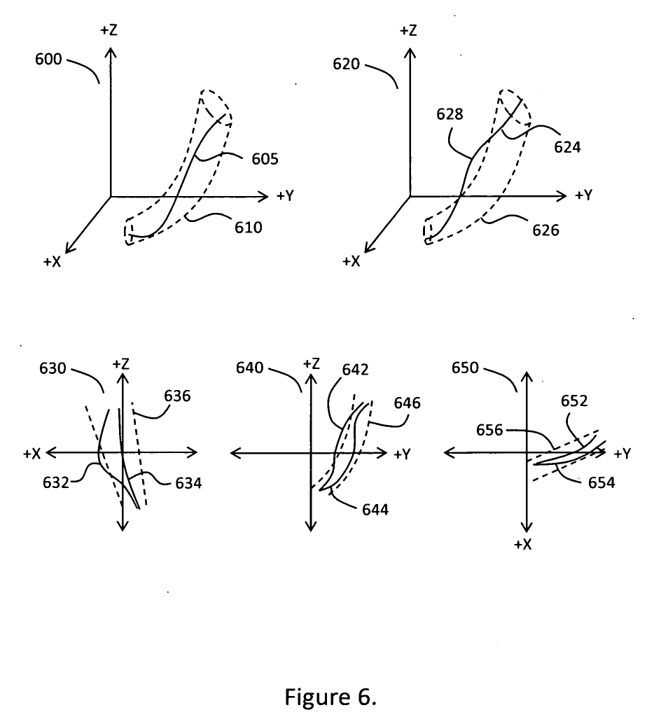

Illustrated in FIG. 6 is a sample set of plots demonstrating two trajectories. With reference to plot 600 in FIG. 6, trajectory 605 lies completely within the bounds 610 while the second trajectory 624 in plot 620 has an interval 628 outside the bounds 626. This is also illustrated in XZ, YZ and XY plane plots 630, 640 and 650. In these three plots, trajectory 605 is projected as line 634, 644 and 654 in plots 630, 640 and 650 respectively. In all these cases, trajectory 605 and the projected trajectories, stay within the bounds illustrated by the dashed lines 636, 646 and 656 in plots 630, 640 and 650 respectively. However, trajectory illustrated with line 624 escapes the bounds 626 and this is represented by the interval 632 in plot 630 and the interval 642 in plot 640. Note that in plot 650, both trajectories remain within the XY plane bounds 656.

This escape from the bounds may or may not cause this repetition to be scored as an unsuccessful or flawed repetition. However, this escape may be useful feedback to the user or trainer. In some cases, this escape could be used to trigger time correlated feedback to specific electrical, mechanical, audio or visual devices. Alternately, this feedback could be displayed in a graphical manner or audio feedback could be employed inform the user of the error. As these plots illustrate, projecting three-dimensional trajectories into planes can provide insight into the specific aspects of a repetition in which a user is failing, and provide directed feedback concerning correcting the action.

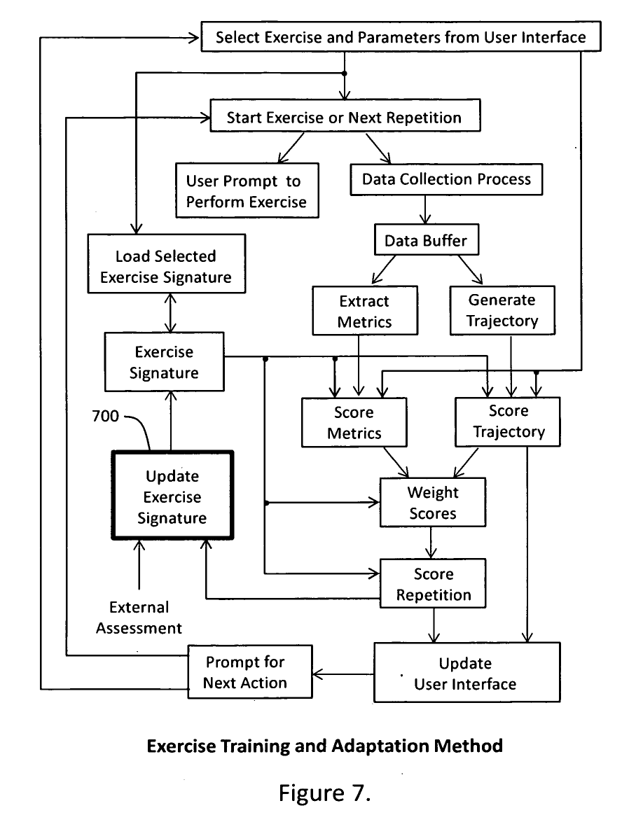

A modification to the Exercise Training Method of FIG. 5 is illustrated in FIG. 7. In this approach scored repetitions are used to update the previously built Exercise Signature. This is represented by the functional block Update Exercise Signature 700 in FIG. 7. The operations performed in Update Exercise Signature function would be a subset of those operations described in relation the Exercise Signature Build Method illustrated in FIG. 3 and described above. The objective of this capability is to enable the Exercise Signature to be adapted to, or purposely track the performance of the user. The input External Assessment may or may not be employed to aid in the process to adapt the existing Exercise Signature. In some cases, it may also be desirable to use the results of performance on one set of exercises to influence the signatures in alternate exercises. This overall process will be referred to as the Exercise Training and Adaptation Method.

Many basic exercises are common to various training and rehabilitation activities. It is also possible to build generic Exercise Signatures for these basic exercises and employ these among a wide class of users. For instance, a library of Exercise Signatures for various classes of individuals, by age, sex, size, could be created for these basic exercises. By use of the Exercise Training and Adaptation Method described in reference to FIG. 7, these generic Exercise Signatures can be readily adapted to specific users.

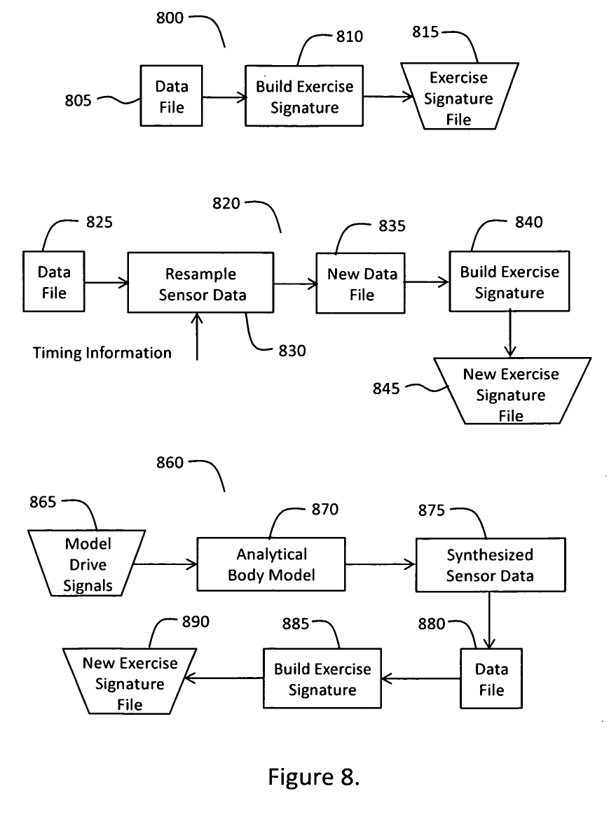

These generic Exercise Signatures may require certain parameter adjustments for specific users. A primary adjustment is time scale. A user may build an Exercise Signature for one time scale version of an exercise. As the user improves in the execution of this exercise, one parameter that is often critical to successful progress is to increase or decrease the speed at which this exercise is performed. Illustrated in FIG. 8 is one method for converting an exercise at one speed or pace to another speed or pace. In flow chart 800, top of FIG. 8, is a high level summary of the method described earlier to generate an Exercise Signature. The process referenced by the functional block Build Exercise Signature 810 represents either the Exercise Signature Build Method or the Exercise Training and Adaptation Method for the generation of an Exercise Signature. This process starts with the Data File 805 containing both raw sensor data and External Assessment inputs. This data is processed by the Build Exercise Signature function 810 to build the Exercise Signature File 815.

Flow chart 820, middle of FIG. 8, illustrates one method for changing the time scale of a given exercise from the time scale captured in the original sensor data, Data File 825. Data File 825 contains the External Assessment data and sensor data from the specific exercise at the originally performed rate 1 (for example one repetition per second). This data is resampled to rate 2 (which for example may be one repetition every 1.5 seconds) in the functional block Resample Sensor Data 830. This new resampling rate is represented by the input Timing Information in flow chart 820. The newly sampled data, together with the original External Assessment data is captured in New Data File 835 which is then processed via Build Exercise Signature 840 to create a New Exercise Signature File 845 representing the original exercise are a rate 2 (1.5 seconds per repetition). The Build Exercise Signature function 840 is either the Exercise Signature Build Method or the Exercise Training and Adaptation Method as previously described.

An alternate method for generating an Exercise Signature File is illustrated in flow chart 860, FIG. 8. In this case an analytical model of the body structure performing the specific exercise is employed. There are a variety of methods for building this model. This model, illustrated as Analytical Body Model 870, represents the dynamics of motion of the various elements of a selected body structure, an arm or leg for example. This model is driven by a set of signals representing specific muscular motions corresponding to a repetition of a specific exercise. This set of signals is represented by the functional block Model Drive Signals 865 in flow chart 860 of FIG. 8. The Analytical Body Model can be augmented with synthetic sensors approximating the placement of real sensors on an actual body. The output of these synthesized sensors are substantially the same as real sensors attached to real body structure performing a repetition of the exercise represented by the Model Drive Signals 865 driving the body structure described by the Analytical Body Model 870. The Synthesized Raw Sensor Data 875 represents the data created by this model. This data, together with synthesized External Assessment data is used to create Data File 880 which can then be employed via Build Exercise Signature 885 to construct a New Exercise Signature File 890.

In a typical Exercise Signature build effort, several sets of Model Drive Signals 865 representing various qualities of repetitions in the performance of the selected exercise, together with the appropriate External Assessment would be constructed and employed.

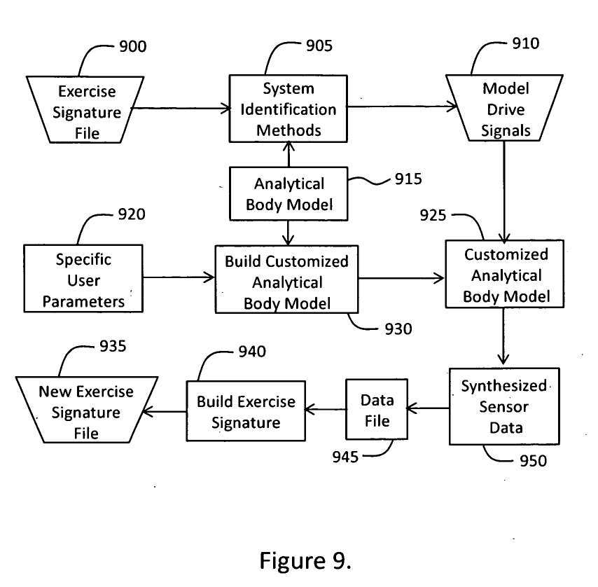

The above described method provides a technique for the construction of generic Exercise Signatures. These Exercise Signatures can be designed, by the specific design parameters of the Analytical Body Model and the Model Drive Signals to accommodate a wide variety of body types, sizes, condition, etc. In some cases, it may be desirable to adapt an Exercise Signature developed for one individual to another individual. One possible version of this process is illustrated in FIG. 9.

This translation process starts with an existing Exercise Signature File 900. Based on information contained in the Exercise Signature File concerning type of exercise, limb(s) involved, etc., an Analytical Body Model 915 can be defined and created. Using System Identification Methods 905 with the trajectory data available from the Exercise Signature File 900, Model Drive Signals 910 can be recovered from the trajectory data. Specific User Parameters 920 containing information regarding the particular individual this exercise is to be customized for is used to modify the original Analytical Body Model 915 via the process Build Customized Analytical Body Model 930. These parameters may include dimensions of the specific user's limb, sex, weight, age, etc. This new Customized Analytical Body Model 925 is now driven by the Model Drive Signals 910 recovered from the original Exercise Signature File 900 trajectory data. Note that the Model Drive Signals 910 may possibly be modified by various Specific User Parameters 920. This customized Analytical Body Model includes synthetic sensors substantially similar to those employed in the generation of the raw data employed to build the original Exercise Signature File 900.

Synthesize Sensor Data 950 is the output of Customized Analytical Body Model 925 driven by Model Drive Signals 910. From information contained in the Exercise Signature File, External Assessment data is also available for each of the several Model Drive Signals 910 recovered from the trajectories in Exercise Signature File 900. This External Assessment data is matched with the Synthesize Sensor Data 950 to build a new Data File 945. The Build Exercise Signature process 940 can now be run on the new Data File 945 to build the New Exercise Signature File 935. This New Exercise Signature file is effectively the original Exercise Signature File customized by the Specific User Parameters 920. What has been accomplished is that an exercise performed by one individual has been translated to the body specifics of a second individual. Methods described in relation to the Signature Modification Methods of FIG. 7 can be employed to further adapt this new Exercise Signature to the new individual.

Consider now general application of this exercise signature building system and training system and possible variations. This system could provide user or trainer defined pacing information from one repetition to the next. Alternately the pacing could be based on the performance of previous repetitions or on the performance on alternate exercises. In other implementations, the system could provide feedback regarding the rate of individual movements in a specific repetition; provide feedback regarding specific changes required to be successful (more/less pronation, for example) and provide other feedback to aid the user in the correct execution of the exercise. This data and could be generated during the execution of an individual repetition and provide nearly instantaneous feedback to the user and/or, provide retrospective feedback and guidance for use in subsequent repetitions.

Results generated by the user in the performance of exercises may be used to count successful and unsuccessful repetitions of various exercises and no information is stored in the system from one use to the next (with the exception of the stored and possibly updated, Exercise Signatures). Alternately, results from one exercise session to the next may be recorded and used in multiple ways. One such use would be to update the number of repetitions, the range of motion, the rate or pace of each repetition, the rest period between repetitions or how often the specific exercise is performed on a daily basis. Another use would be to advance the user through different exercise routines as a function of recorded results, time of day, day of the month, etc. For instance, as a user's range of motion or strength increased, the system could observe these results from the recorded data and select alternate Exercise Signatures requiring the user to increase weight and/or alter the range of motion in order to record a successful repetition of a given exercise. Recorded data could also be used to alter the ordering of exercises or the specific exercises practiced from one session to the next.

An additional use of recorded exercise performance information would be to forward results, and possibly measured sensor data to health professionals, trainers or other 3^{rd} parties to review and monitor progress and update exercise routines, programs, etc. In the same way, the system could also provide alerts to 3^{rd} parties regarding incorrect use or potentially less than desirable situations and other information useful for managing effective rehabilitation or training. These same remote connection capabilities could also allow 3^{rd} parties to modify Exercise Signatures, alter training regimes, pacing, etc., to aid the user in accomplishing specific goals.

Processing elements contained in central processing system 200 of FIG. 2 may be any integrated circuit device configured for a particular purpose. As such, the central processing system 200 in FIG. 2 may be any application specific integrated circuit (ASIC), microprocessor, or other logic device known in the art or developed in the future. Data storage system 210 in FIG. 2 may be a form of removable storage, may be dedicated hardware of the system or some combination of both and may be of any currently available storage media currently known in the art or developed in the future.

No specific bussing technologies for bus 225 or specific communications methods for Communications System 215 have been identified in this document. The applications and methods taught in this patent application are substantially independent of these technologies. Consequently, this system may employ virtually any bussing and communications methods currently available or those developed in the future.

Several references have been made to techniques associated with pattern recognition technologies and methods. Those skilled in the art of pattern recognition will recognize multiple methods in which the training, measuring and scoring processes can be implemented. References to specific techniques have not been made since the specifics of these methods are substantially independent of the applications taught in this patent application.

The previous discussion is not intended to limit the specific numbers, types and physical or logical arrangements of sensors, specific data rates, bussing or communications systems. References to specific techniques are used only as a means to explain an example of the art. Those skilled in these methods are aware of many alternate methods that can be employed.

In summary, systems, devices, and methods configured in accordance with exemplary embodiments relate to:

A physical structure of one or more sensors coupled in some communications network to a data processing system in which the data processing system is connected in various ways to a user interface, data storage and communications systems which is intended to collect data regarding the motions of a body performing a physical movement. The sensors are attached to a body in some manner which substantially maintains these sensors in a fixed physical relationship to the body and to each other. The collected data is used to either generate reference Exercise Signatures for the future measurement and scoring of subsequent body motion, or to be used in the measurement and scoring of these body motions relative to the previously generated Exercise Signatures. In certain embodiments, the sensors may be one or more of an angular or linear accelerometer, gyroscope, tachometer, angular resolver, pressure, acoustic, temperature, magnetic, optical, torsion, tension or force measuring devices.

The sensor and physical structure as described above in which collected data, together with external measures, are used to generate Exercise Signatures which represent one or more grades of performance of a particular body motion or exercise.

The sensor and physical structure as described above in which collected data are compared in some manner to previously generated Exercise Signatures to measure or score the performance of a specific execution of an exercise or other specific body motion.

The sensor and physical structure as described above in which results of the scored exercises or body motions are provided to the exerciser in some manner. This feedback may be visual, audio, mechanical, olfactory, by taste or electrical in nature.

The sensor and physical structure as described above in which results of the scored exercises or body motions are provided to the user in some manner during the execution of a specific repetition in order to guide the performance of this repetition. This feedback may be visual, audio, mechanical, olfactory, by taste or electrical in nature.

The sensor and physical structure as described above in which results of the scored exercises or body motions are employed to modify the particular Exercise Signature, select alternate exercises, alter pace, quantity, form, weight or other relevant elements of an exercise or body motion. This feedback may be visual, audio, mechanical, olfactory, by taste or electrical in nature.

The sensor and physical structure as described above in which collected data, exercise results, statistics or other measures are stored and/or communicated to 3^{rd} parties. This communication may be immediate or delayed. This communications may also allow 3^{rd} parties to monitor performance in real-time to provide immediate feedback on performance or to enable changes in exercise parameters associated with specific Exercise Signatures.

The sensor and physical structure as described above in which existing Exercise Signatures can be adapted to changing user requirements.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention.

1. An appliance device comprising:
a data processing system consisting of data processing devices;
data storage devices consisting of volatile and non-volatile memory systems coupled to the data processing devices and used for storing data files, collected data, intermediate processor data and for storing software program instructions used by the data processor;
an array of one or more sensors coupled to the data processor and configured to generate an output measuring the sequence of motions of the body structure to which the sensors are attached;
said array of sensors attached on a particular structure of an animal body and this attachment method maintaining these sensors in a substantially fixed position on the animal body for the duration of the exercise period;
data processing methods enabling the building of exercise signature data structures substantially representing the specific body structure motions corresponding to a particular exercise from a combination of external assessment data representing a quality measure of each of several repetitions of the particular exercise and sensor data collected by said array of sensors attached to a particular body structure, said sensor data collected during the execution of several repetitions of the particular exercise;
data processing methods to collect data generated by said array of sensors attached to a particular body and to compare this data to the exercise signature data structures representing this specific exercise and generate measures of the quality of similarity between each of the several repetitions of a specific exercise and the exercise signature data structures representing repetitions of this specific exercise;
data processing methods employing said measures of the quality of similarity to score each of the several repetitions of the specific exercise in order to provide constructive feedback to the user or other parties concerning the performance of each of the several repetition of the specific exercise;
a user interface providing a means to provide said feedback to the user of this appliance device and other parties;
a user interface providing the ability for the user and other parties interfaced to this appliance device to select various specific exercises, update parameters concerning these specific exercises, control general operations of this appliance device; and
a user interface capable of providing means to provide stimulus for audio, visual, mechanical or electrical feedback to the user of this appliance device.

2. The appliance device of claim 1, augmented with the capability to generate, record, store and analyze various statistics concerning the use of this appliance device.

3. The appliance device of claim 1, augmented with communications systems enabling this appliance device to communicate with 3^{rd} party data processing platforms for the purposes of transferring software, original or processed sensor data, statistical data, data files, updating and managing the appliance device, provide remote user interface functionality and enabling means by which specific 3^{rd} party user inputs can be relayed to the appliance device.

4. The appliance device of claim 1, augmented with data processing methods enabling the capability to adapt a specific exercise signature data structure with new data collected by said array of sensors measuring repetitions this specific exercise.

5. The appliance device of claim 1, augmented with removable data storage devices enabling the transfer of data between this appliance device and other data processing platforms.

6. The appliance device of claim 1, augmented with data processing methods enabling the capability to transform a reference exercise signature data structure to a specific user performing substantially the same exercise as represented in the reference exercise signature data structure.

7. An appliance device comprising:
a data processing system consisting of data processing devices;
data storage devices consisting of volatile and non-volatile memory systems coupled to the data processing devices and used for storing data files, collected data, intermediate processor data and for storing software program instructions used by the data processor;
an array of one or more sensors coupled to the data processor and configured to generate an output measuring the sequence of motions of the body structure to which the sensors are attached;
said array of sensors attached on a particular structure of an animal body and this attachment method maintaining these sensors in a substantially fixed position on the animal body for the duration of the exercise period;
communications systems enabling one appliance device to communicate and share data and other user specific information with substantially similar appliance devices that may be either on the same user's body or on alternate user's bodies;
data processing methods enabling the building of exercise signature data structures substantially representing the specific body structure motions corresponding to a particular exercise from a combination of external assessment data representing a quality measure of each of several repetitions of the particular exercise and sensor data collected by said array of sensors attached to a particular body structure and data collected by a substantially similar appliance device attached to possibly a second body structure, said sensor data collected during the execution of several repetitions of the particular exercise;
data processing methods to collect data generated by said array of sensors attached to a particular body structure and data collected by a substantially similar appliance device attached to a possibly second body structure while said body structures are performing repetitions of a specific exercise and to compare this data to the exercise signature data structures representing this specific exercise and generate measures of the quality of similarity between each of the repetitions of a specific exercise and the exercise signature data structures representing repetitions of this specific exercise;
data processing methods employing said measures of the quality of similarity to score each of the several repetitions of the specific exercise in order to provide constructive feedback concerning the performance of each of the several repetition of the specific exercise;
a user interface providing a means to provide said feedback to the user of this appliance device and other parties;
a user interface providing the ability for the user and other parties interfaced to this appliance device to select various specific exercises, update parameters concerning these specific exercises, control general operations of this appliance device;
a user interface capable of providing means to provide stimulus for audio, visual, mechanical or electrical feedback to the user of this appliance device.

8. The appliance device of claim 7, augmented with the capability to generate, record, store and analyze various statistics concerning use of this appliance device.

9. The appliance device of claim 7 augmented with communications systems enabling this appliance device to communicate with 3^{rd} party data processing platforms for the purposes of downloading software, data structures and data files, updating and managing the device, uploading data or results, emulate the user interface, provide additional user interface functionality and enabling means by which specific 3^{rd} party user inputs can be relayed to the device;

10. The appliance device of claim 7, augmented with data processing methods enabling the capability to adapt specific exercise signature data structures with new data collected by said array of sensors measuring repetitions this specific exercise.

11. The appliance device of claim 7, augmented with removable data storage devices enabling the transfer of data between this appliance device and other data processing platforms.

12. The appliance device of claim 7, augmented with data processing methods enabling the capability to transform a reference exercise signature data structure to a specific user performing substantially the same exercise as represented in the reference exercise signature data structure.
